(19) United States
(12) Patent Application Publication
(10) Pub. No.: US 2011/0033397 A1
(43) Pub. Date: Feb. 10, 2011

(54) Anti-Aging Compositions Comprising Menyanthes Trifoliata Leaf Extracts And Methods Of Use Thereof

(76) Inventors: Hugo Corstjens, Maaseik (BE); Lieve Declercq, Ekeren (BE); Ilse Sente, Zonhoven (BE); Annelise Lobstein, Lipsheim (FR); Bernard Weniger, Strasbourg (FR); Robert Anton, Strasbourg (FR)

Correspondence Address: THE ESTEE LAUDER COS, INC, 155 PINELAWN ROAD, STE 345 S, MELVILLE, NY, 11747, US

(21) Appl. No.: 12/911,438

(22) Filed: Oct. 25, 2010

Related U.S. Application Data
(63) Continuation of application No. 11/461,093, filed on Jul. 31, 2006, now abandoned.

(51) Int. Cl. A61K 8/97; A61Q 17/04; A61K 8/368; A61K 8/365; A61K 8/49

(52) U.S. Cl. 424/59; 424/774; 514/568; 514/570; 514/456; 514/457

(57) ABSTRACT

An anti-aging composition comprising a skin-beneficial amount of actives identified in Menyanthes trifoliata leaf, wherein the actives are inhibitors of one or more of MMP-1, 2 and 9 and/or scavengers of peroxynitrite. Also disclosed are methods of using such a composition, which include treating the skin for signs of chronological or pre-mature aging.

This application is a continuation of U.S. Ser. No. 11/461,093, filed Jul. 31, 2006, and claims benefit therefrom.

### FIELD OF THE INVENTION

The present invention relates to anti-aging skin care compositions and methods. In particular, the present invention relates to novel anti-aging compositions comprising Menyanthes trifoliata leaf extracts and methods of treating the signs of chronological or pre-mature aging.

### BACKGROUND OF THE INVENTION

### Free Radical Damage

It is well known that the production of corrosive reactive oxygen species (ROS) in human skin cells occurs as a result of normal cell function, but cells naturally contain anti-oxidants that reduce the free radicals, thereby preventing or limiting damage to the cell. A number of ROSs have been identified and these include the hydroxyl radical, hydrogen peroxide, peroxide, singlet oxygen, superoxide and nitric oxide. Pathological production of reactive oxygen species (a.k.a. oxidative stress) also occurs in human skin cells, wherein unchecked levels of ROSs in a cell damage cell components and impair cell function. Sufficiently damaged cells may exhibit decreased energy production, senescence, mutations in the mitochondrial DNA, altered functioning of the cell membrane and defective apoptosis mechanisms. Ultimately, cells so damaged accumulate in the surrounding tissue (i.e. skin) and have a detrimental effect on the tissue and the individual. In particular, skin tissue may develop a decreased capacity to heal or repair itself and collagen production may be significantly decreased. Ultimately, these effects may manifest on the exterior as lines, wrinkles, blemishes and other telltale signs of aging.

In the skin, naturally occurring anti-oxidants decrease with age, such that the cells normal defense mechanism may not keep up with production of free radicals. This imbalance is a result of genetic factors and the visible manifestations in the skin that result are may be termed chronological aging. On the other hand, an imbalance may also result from or be exacerbated by an overproduction of free radicals, induced by external factors. For example, it is well known that UV exposure is capable of generating quantities of ROSs that cannot be neutralized by the cells natural defense mechanism before damage is incurred. As a result, skin cells with various types of damage accumulate in the tissue. The collective detrimental effects of UV exposure are known as photoaging, as opposed to chronological aging. Other external factors may create a pathological condition in the skin of excessive free radicals; smoking, pollution, psychological stress, dermatological disorder, vascular disorder, allergy, etc.

A signature sign of aging skin, regardless of the etiology, is loss of elasticity resulting from reduced production of collagen and the degradation of existing collagen. Collagens are fibrous structural proteins and a main component of the extracellular matrix of connective tissue. Collagen contributes to the strength and elasticity of human skin, and its degradation leads to changes in the appearance and/or function of the skin, such as wrinkles, including fine, superficial wrinkles and coarse, deep wrinkles, lines, crevices, bumps, enlarged pores, scaliness, flakiness loss of skin elasticity, sagging (including puffiness in the eye area and jowls), loss of skin firmness, compromised barrier properties, discoloration (including undereye circles), blotching, sallowness, mottled pigmentation, age spots, freckles, keratoses, abnormal differentiation, hyperkeratinization, elastosis, telangiectasia and other histological changes in the stratum corneum, dermis, epidermis, the skin vascular system.

Numerous attempts have been made to reduce the detrimental effects of UV radiation on the skin. Sunscreens are commonly used to prevent photoaging of skin by sunlight. Sunscreens are topical preparations that contain ingredients that absorb, reflect and/or scatter UV light. Some sunscreens are based on opaque particulate materials including zinc oxide, titanium oxide, clays, and ferric chloride. However, because such preparations are visible and occlusive, many people consider those opaque formulations to be cosmetically unacceptable. Other sunscreens contain chemicals such as p-aminobenzoic acid (PABA), oxybenzone, dioxybenzone, ethylhexyl-methoxy cinnamate, octocrylene, octyl methoxycinnamate, and butylethoxydibenzoylmethane that are transparent or translucent on the skin. While these types of sunscreens may be more acceptable cosmetically, they are still relatively short-lived and susceptible to being removed by washing or perspiration. Moreover, there is a continuing trend in the art to provide naturally-derived skin care ingredients for application to the skin. Despite the widespread use of sunscreens, photoaging continues to be a serious health issue.

### MMP-1, 2 and 9 Imbalance

The extracellular matrix (ECM) of the skin imparts strength and integrity to the skin. Matrix metalloproteinases (MMPs), are proteases that are capable of dissolving peptide bonds, thereby degrading the collagen that is a prevalent component of the ECM. MMPs play a role in normal degradation and remodeling as part of the skin's self maintenance. However, over-activation of MMPs leads to or exacerbates pathological conditions resulting in loss of tissue function and/or structure. There are various types of MMPs, but recently considerable attention has been given to the role of specific MMPs in the field of remodelling of the skin extracellular matrix, wound healing, inflammation and oxidative stress, including oxidative stress associated with UV exposure (see, for example, “Metalloproteinase Inhibitors” Thibodeau, A., Cosmetics & Toiletries, 2000; 115: 75-80). Three MMPs identified as MMP-1, MMP-2 and MMP-9 are particularly associated with the extracellular matrix of the skin and play a role in normal and pathological tissue remodeling. For two reasons then, MMP-1, 2 and 9 are of particular interest. First, because the substrates against which these MMPs act are the very structural components of the skin and second, because the skin is continually exposed to the agents that trigger pathological states of these MMPs, namely, inflammation, oxidative stress and UV exposure. Selective inhibition of these three MMPs may therefore prove to be beneficial and more efficient compared to general targeting of metalloproteinases.

A main component of the skin extracellular matrix comprises glycoproteins and most glycoproteins in the extracellular matrix are collagens. Enzymatic degradation of collagens by MMP-1 (a.k.a. interstitial collagenase) has been known for decades. MMP-1 is important for its ability to degrade triple-helix collagens. MMP-1 cleaves preferentially collagen type I and thus plays an important role in the degradation of dermal collagen and wound healing (see “Induction of matrix metalloproteinase-1 in in vitro experimental wound model using a novel three-dimensional culture system” Kan, et al., Eur J Dermatol 2001 March-April; 11(2):112-6). Strong induction of MMP-1 is also found in smokers compared to non-smokers (see “Matrix metalloproteinase-1 and skin ageing in smokers” Lahmann, et al., Lancet 2001 Mar. 24; 357(9260):935-6; and “Skin aging induced by ultraviolet exposure and tobacco smoking: evidence from epidemiological and molecular studies” Yin, et al., Photodermatol Photoimmunol Photomed 2001 August; 17(4):178-83).

Both MMP-2 (gelatinase A or 72 kDa type IV collagenase) and 9 (gelatinase B or 92 kDa type IV collagenase) degrade Type IV collagen, which is associated with the basal lamina, which supports the epithelium in the outer skin. Both MMP-2 and MMP-9 have been shown to be activated by oxidative stress (see, “Oxidative stress regulates collagen synthesis and matrix metalloproteinase activity in cardiac fibroblasts” Siwik, et al., Am J Physiol: Cell Physiol 2001 January; 280(1):C53-60). They are also known to be expressed during wound healing (see “Expression of matrix metalloproteinase-2 and -9 during early human wound healing” Salo, et al., Lab Invest 1994 February; 70(2):176-82 and “Functional overlap between two classes of matrix-degrading proteases in wound healing” Lund, et al., EMBO J 1999; 18(17):4645-56). In addition MMP-9 is also upregulated during inflammation (“TNFα Upregulated MMP-9 Secretion by Human Keratinocytes Via MAPK and NF-κB Activation” Holvoet, et al., presentation at ESDR, Geneva, 2002), while MMP-2 plays a major role in specific degradation of basement membrane and disruption of basement membrane integrity (see, “Critical appraisal of the use of matrix metalloproteinase inhibitors in cancer treatment.” Zucker, et al., Oncogene 2000 Dec. 27; 19(56): 6642-50).

Furthermore, it has been reported that MMP-1, 2, and 9 may be activated by exposure to UV radiation. Specifically, MMP-1 and 2 are activated by UVA, while MMP-1 and 9 are activated by UVB (see, “Metalloproteinase Inhibitors” Thibodeau, A., Cosmetics & Toiletries, 2000; 115: 75-80). The activation of MMP-2 UVA was noted, in vitro. It has been reported that UVB exposure causes dermal fibroblasts to over-produce MMP-1 (see “Direct Role of Human Dermal Fibroblasts and Indirect Participation Of Epidermal Keratinocytes In MMP-1 Production After UVB Irradiation” Fagot, et al., Arch Dermatol Res, 2002: 293: 576-83).

MMPs are synthesized in an inactive form (i.e. proMMPs a.k.a. zymogens) and must be activated before collagen degradation can occur. Once activated, MMPs are regulated by tissue inhibitors of metalloproteinase (or TIMPs), which can block MMP enzymatic activity. In a model of healthy human skin, MMP activation and MMP inhibition occur in concert to maintain the correct level of collagen breakdown as part of the skin's self maintenance. In fact, throughout life, the balance between MMP activation and inhibition gradually tips toward MMP activation. Tipping of the balance occurs as a result of the inherent (genetic) aging process, even apart from exogenous factors. With age, the rate of MMP activation increases, while the rate of production of TIMP-1 and TIMP-2 decreases. Thus, it appears quite inevitable, that age brings on a loss of integrity of the extracellular matrix and associated visible signs of aging. Additionally, however, even in younger skin, the balance between MMP activation and inhibition may be tipped toward activation by exogenous factors, such as oxidative stress, UV exposure, inflammation and tobacco use. As noted, chronic exposure to any of these causes activation of one of more of MMP-1, 2 and 9. This type of activation lies outside of the normal tissue remodeling mechanism and as such is not perfectly well regulated by a corresponding recruitment of MMP inhibitors. This imbalance has detrimental effects on the human skin, visibly manifesting as signs of premature aging.

### Peroxynitrite Damage and MMP Imbalance

Two of the reactive oxygen species noted above, superoxide and nitric oxide, react, under pathological conditions, to form peroxynitrite, which is itself a potent reactive species. Unchecked, peroxynitrite is known to cause a number of detrimental effects within a cell. These include DNA lesions, inhibition of cell proliferation and, in sufficient concentrations, cytotoxicity. Added to these nasty effects of peroxynitrite is the observation (in vitro) that peroxynitrite activates MMPs and proMMPs (see “Enhanced Vascular Permeability In Solid Tumor Involving Peroxynitrite And Matrix Metalloproteinases” Wu, et al., Jpn J Cancer Res, 2001; 92: 439-51).

Thus, the situation is such that UV exposure causes high concentrations of toxic free radicals that cause an array of damage to the human skin, including decreased production of new collagen. In addition UV exposure directly causes an imbalance in MMP production, leading to excessive breakdown of existing collagen. And finally, to make matters worse, two of the UV induced free radicals react to form peroxynitrite, which further encourages MMP activation leading to even more collagen loss. Scavenging free radicals, alone, would provide some protection for the skin. Likewise, inhibiting overproduction of MMPs-1, 2 and 9, absent peroxynitrite scavenging, would provide some protection for the skin. But the most protection against the vicious cycle of MMP and peroxynitrite overproduction is to attack both pernicious factors. Therefore, there remains a need for a novel composition capable of protecting the skin from collagen decline by inhibiting skin-specific, UV-specific MMPs (1, 2 and 9) and removing peroxynitrite from an affected site. While not wishing to be bound by any one theory, it is believed that reduction and or inhibition of skin-specific, UV-specific MMPs (1, 2 and 9) and the removal of peroxynitrite from an affected site, will prove highly beneficial for combating the loss or decline of collagen and for preventing, reducing, forestalling, reversing or treating the signs of aging, noted above.

### Menyanthes Trifoliata

Menyanthes trifoliata (a.k.a. bogbean, buckbean, bitter worm and others) is common in the marshes and bogs of Europe, but can also be cultivated in shallow waters. It is reported to have been used as an oral supplement for treating the liver, gall bladder, blood production dysfunction, as well as headaches, rheumatism, scurvy, fever, trigeminal neuralgia, gastritis and general fatigue. Menyanthes trifoliata is reported to have a marked stimulating action on the digestive juices and on bile flow. As such, it aids in debilitated states that are due to sluggish digestion, indigestion and problems of the liver and gall-bladder. Although having a bitter taste, Menyanthes trifoliata is also used as a tea to cure dyspepsia and a torpid liver. Menyanthes trifoliata has also been recommended as an external application for dissolving glandular swellings. Curiously, however, topical applications have been reported to cause irritation and congestion. Its use has been reported to cause headache with obscured vision and fever.

U.S. Pat. No. 5,529,769 discloses cosmetic composition containing betulinic acid. The betulinic acid, it is disclosed, may come from a number of plant sources, of which Menyanthes trifoliata is mentioned. The reference also lists a number of solvents that may be used to extract betulinic acid. However, the reference fails to specify which solvent or solvents may be used on Menyanthes trifoliata to extract betulinic acid. Even more critical, the reference fails to identify the portion or portions of the plant from which betulinic acid may be extracted. On this point, see “Biologically Active Pentacyclic Triterpenes And Their Current Medicine Signification” Patocka, J., Journal of Applied Biomedicine, 1:7-12, 2003 (ISSN 1214-0287), which states, “Betulinic acid is found in many plant species. Its content, however, is low. Menyanthes trifoliata, a bog plant, is the rare exception (Huang et al. 1995). Its underground parts contain marked amounts of free betulinic acid . . . ” “Underground parts” refer to the root and rhizome of Menyanthes trifoliata. “Underground parts” specifically does not refer to the leaves, which is the concern of the present invention. See also, “Dr. Duke's Phytochemical and Ethnobotanical Databases” (a website of the US Agricultural Research Service, accessed at: http://www.ars-grin.gov/duke/). In this database, the entry for betulinic acid confirms that betulinic acid is found in the root and rhizome of Menyanthes trifoliata, and not in the leaves.

Applicants analyzed compositions made with Menyanthes trifoliata leaf extract (supplied by Monteloeder), for the presence of betulinic acid. The results of an HPLC analysis confirm the absence of betulinic acid in the composition and therefore, the absence of betulinic acid in the Menyanthes trifoliata leaf extract, at least to the detection limits of the equipment used (3 μg of per gram of product). Therefore, U.S. Pat. No. 5,529,769 does not disclose or even suggest a composition comprising Menyanthes trifoliata leaf extract nor their use in treatment of aging skin. Therefore, in the '769 reference there is no teaching or suggestion of an anti-aging composition comprising a skin-beneficial amount of certain actives identified in Menyanthes trifoliata leaf extracts.

U.S. Pat. No. 6,482,857 U.S. Pat. No. 6,124,362 and U.S. Pat. No. 6,451,777 all discloses compositions or methods for regulating hair growth containing betulinic acid. The betulinic acid, it is disclosed, may come from Menyanthes trifoliata. The method of extraction from Menyanthes trifoliata is not disclosed and the portion of the plant from which betulinic acid may be extracted is not identified. Given that the prior art identifies the root and the rhizome of Menyanthes trifoliata as sources of betulinic acid, none of these references teach or suggest a cosmetic composition comprising a skin-beneficial amount of certain actives identified in Menyanthes trifoliata leaf extracts.

JP 07-061916 discloses a skin external agent comprising kojic acid and one or more plant extracts, of which buckbean (Menyanthes trifoliata) is mentioned. The composition is said to have “excellent elasticity-restoring activity on aged skin by using kojic acid and/or its derivative in combination with a specific plant extract and synergistically enhancing the cell proliferation activity of kojic acid and/or its derivative.” Like the preset invention, the reference is specifically concerned with reversing the loss of skin elasticity due to UV exposure. Unlike the present invention, the focus in this reference is on “raising a cell proliferation operation of kojic acid or a kojic acid derivative in multiplication [i.e. synergistically] . . . by using the extractives of specific vegetation together.” The reference clearly implies that by themselves, the specified plant extracts, Menyanthes trifoliata, in particular, do not have any stated activity. Rather, the combination of a plant extract with kojic acid enhances some activity of the kojic acid. Therefore, in this reference there is no teaching or suggestion of a cosmetic composition comprising a skin-beneficial amount of actives derived from Menyanthes trifoliata leaf extracts, the actives selected from the group consisting of phenolic acids, coumarins and flavonoids.

To date, anti-aging compositions comprising a skin-beneficial amount of actives identified in Menyanthes trifoliata leaf extracts, wherein the actives are selected from phenolic acids, coumarins, flavonoids and mixtures thereof, are unknown in the art. Furthermore, unknown is a method of reducing the signs of aging on the skin, comprising applying a skin-beneficial amount of an extract of Menyanthes trifoliata leaf.

### SUMMARY OF THE INVENTION

The present invention includes an anti-aging composition comprising skin-beneficial amounts of an extract of the Menyanthes trifoliata leaf. The extract comprises an amount of certain actives that are effective at inhibiting the activity of matrix metalloproteinases-1, 2 and 9, and/or effective at scavenging peroxynitrite. These actives include, but may not be limited to specific phenols, coumarins and flavonoids.

### DETAILED DESCRIPTION

Except in operating and comparative examples, or where otherwise explicitly indicated, all numbers in this description indicating amounts or ratios of material or conditions of reaction, physical properties of materials and/or use are to be understood as modified by the word “about.” All amounts are by weight of the final composition, unless otherwise specified.

Compositions herein described are particularly useful in methods of treating signs of aging. As used herein, “treating the signs of aging” includes preventing, reducing, forestalling, reversing or treating the signs of aging mentioned above, whether the cause be chronological or pre-mature aging.

As used herein, “skin beneficial” means that the extract comprises an amount of certain actives that are effective at inhibiting the activity of matrix metalloproteinases-1, 2 and 9, and/or effective at scavenging peroxynitrite.

The present invention is predicated on the observation that extracts of the leaves of Menyanthes have a surprising ability to protect skin cells against the damaging effects of UV radiation. Specifically, it has been surprisingly discovered that extracts of Menyanthes leaves effectively inhibit specific matrix metalloproteinases implicated in UV damage, while also scavenging reactive oxygen species (ROS). The ROSs in question are related to proMMP activation, but are also known to degrade the skin via oxidative stress, thus posing a double threat to the skin. Specifically, while not wishing to be bound by any one theory, it is believed that such plant extracts protect against UV-induced skin damage and related oxidative stresses, by inhibiting and/or reducing MMPs-1, 2 and 9 that degrade the dermal collagen, while also scavenging peroxynitrite. As such, the compositions of the present invention would provide a double benefit in that the compositions reduce MMPs as well as scavenge ROSs.

As experiments show (see Examples 3) the primary active or effective components, capable of inhibiting MMP-1, 2 and 9, are specific phenolic acids, flavonoids and coumarins extracted from the leaves of Menyanthes trifoliata. Furthermore, it is shown herein, that the specific, active phenolic acids present in the Menyanthes trifoliata leaf extracts are ferulic acid and protocatechuic acid. Specific, active flavonoids are quercetin, iso-quercetrin and rutin. Specific, active coumarins are scoparone and scopoletin.

While components of Menyanthes have been reported as having various types of biological activity, it was unexpected that Menyanthes trifoliata leaf extracts would exhibit specific MMP-1, 2 and 9 inhibition activity and that the specifically named phenolic acids, flavonoids and coumarins would be primarily responsible for such. In addition, although the specific phenolic acids, flavonoids and coumarins are herein shown to be the principle active components in achieving inhibition of MMP-1, 2 and 9, additional components, although not necessarily very effective on their own, may be present in the plant extracts that can have some contributory activity.

In the preferred embodiment, an extract of Menyanthes trifoliata L. is used. It is expected that other species of Menyanthes may also prove useful, including, cristata Roxb., hydrophylla Lour., indica, meridionalis Willd. ex Griseb, nymphoides L., ovata L. f, pumila Douglas ex Griseb., punctata Muhl. ex Griseb and trachysperma Michx and combinations thereof. In the preferred embodiment, Menyanthes trifoliate L. is used, although other subspecies may also prove useful, including, but not limited to trifoliata fo. Brevistyla Aver., trifoliata var. minor Michx. Ex Raf, trifoliata subsp. Trifoliata, trifoliate var. trifoliata and trifoliata subsp. Verna.

“Menyanthes trifoliata extract” is a generic term describing a number of different chemical compositions that may contain several different active components. Numerous extracts are commercially available, and any one of those may prove useful in the present invention. However, particularly preferred for use is a Menyanthes trifoliata L. extract available from Monteloeder in Spain. It will be understood that the term “Menyanthes extract” as used herein shall encompass not only a Menyanthes extract per se, but also a composition to which one or more of the active components such as noted herein, are added. Such added active components may be from synthetic or natural sources, either from Menyanthes or from material other than Menyanthes, in amounts equivalent to those described in the use of the Menyanthes extract.

Menyanthes extracts containing the specific active phenolic acids, flavonoids and coumarins, are most easily obtained by contacting the plant part with a suitable solvent or solvent(s), according to methods known in the art. The choice of the solvent should be made based on the properties of the active ingredient that is to be extracted. Ultimately, the extract may be isolated from the solvent. Particularly preferred solvents are alcoholic, ethyl acetate and dichloromethane. As the examples show, these solvents produce extracts of Menyanthes trifoliata that possess the specific active components needed to inhibit MMP-1, 2 and 9 and scavenge peroxynitrite. The concentration of solvent may be adjusted by a person skilled in the art and the extraction may be repeated on the same sample to increase the yield. The alcoholic, ethyl acetate or dichloromethane extracts will contain elements other than the specific active components. Nevertheless, the extracts may be used without further refinement or, alternatively, the specific active components may be isolated from the extract.

Based on total weight of a composition according to the present invention, the composition will comprise from 0.001 to 15 wt % of the active components, whether they are added in extract or isolated form. Where cost or other factors dictate, preferable concentrations range from 0.01 to 10 wt %, or most preferably from 0.1 to 5 wt % of the active components, whether they are added in extract or isolated form. To achieve broad spectrum efficacy, it is preferable that compositions according to the present invention comprise active components from at least two of phenolic acids, flavonoids and coumarins. Most preferably, compositions according to the present invention comprise active components from all three of phenolic acids, flavonoids and coumarins. The preferred concentration of specific phenolic acids is 0.001 to 5.00 wt-%. The preferred concentration of specific flavonoids is 0.001 to 5.00 wt-%. The preferred concentration of specific coumarins is 0.001 to 5.00 wt-%.

When the active components are added in extract form, the concentration of Menyanthes trifoliata extract in the composition depends on the concentration of the actives in the extract. Typically, the alcoholic extract, ethyl acetate extract, dichloromethane extract or combinations thereof may be used in an amount from 0.01 to 20% of the composition to provide a skin beneficial concentration of active components. Nevertheless, larger concentrations are not outside the scope of this invention.

In an alternate embodiment, the present invention includes a sunscreen. Suitable sunscreens include water soluble sunscreens (such as Eusolex 232); oil soluble sunscreens (such as octyl methoxycinnamate); inorganic sunscreens (such as titanium dioxide, zinc oxide) and organic sunscreens (such as camphor derivatives, cinnamates, salicylates, benzophenones, triazines, PABA derivatives, diphenylacrylate derivatives, and dibenzoylmethane derivatives.) The amount will vary depending on the formulation and the performance desired. The sunscreen may be used in an amount from 0.1% to 50% by weight of the composition. Preferably, the sunscreen is used in an amount from 1% to 40% and most preferably, an amount from 5% to 30%.

The composition further comprises a cosmetically acceptable vehicle that is suitable for topical application to skin, hair and/or nails. Cosmetically acceptable vehicles are well known in the art and are selected based on the end use of the application. For example, vehicles of the present invention include, but are not limited to, those suitable for application to the skin. Such vehicles are well known to those of ordinary skill in the art, and can include one or more compatible liquid or solid filler diluents or vehicles which are suitable for application to the skin. The exact amount of vehicle will depend upon the level of any other optional ingredients that one of ordinary skill in the art would classify as distinct from the vehicle (e.g., other active components). In compositions of the present invention, the vehicle may comprise from about 75 to about 99.99 wt % of the composition.

The vehicle and the compositions herein, may be formulated in a number of ways, including but not limited to emulsions. For example, suitable emulsions include oil-in-water, water-in-oil, water-in-oil-in-water, oil-in-water-in-oil, and oil-in-water-in-silicone emulsions. Preferred compositions comprise an oil-in-water emulsion.

The compositions of the present invention can be formulated into a wide variety of product types, including shampoos, creams, waxes, pastes, lotions, milks, mousses, gels, oils, tonics and sprays. Preferred compositions are formulated into lotions, creams, gels, shampoos and sprays. These product forms may be used for a number of applications, including but not limited to, hand and body lotions, cold creams, facial moisturizers, anti-acne preparations, topical analgesics, color cosmetics including foundations, eyeshadows, lipsticks and the like. Any additional components required to formulate such products vary with product type and can be routinely chosen by one skilled in the art.

### Other Components

The formulation may also comprise components that are chosen depending on the carrier and/or the intended use of the formulation. Additional components include, but are not limited to antioxidants, chelating agents, emulsion stabilizers, preservatives, fragrances, flavoring agents, humectants, waterproofing agents, water soluble film-formers, oil-soluble film formers, moisturizing agents, such as cholesterol, cationic polymers, anionic polymers, vitamins, propellants and the like.

The compositions may encompass one or more additional active components, to render either a cosmetic or pharmaceutical composition. Examples of useful actives include, but are not limited to, those that improve or eradicate age spots, keratoses and wrinkles; analgesics, anesthetics, anti-acne agents, antibacterials, antiyeast agents, antifungal agents, antiviral agents, antidandruff agents, antidermatitis agents, antipruritic agents, antiemetics, antihyperkeratolytic agents, anti-dry skin agents, antiperspirants, antipsoriatic agents, antiseborrheic agents, hair conditioners and hair treatment agents, antiaging agents, antiwrinkle agents, antiasthmatic agents and bronchodilators, sunscreen agents, antihistamine agents, depigmenting agents, wound-healing agents, vitamins, corticosteroids, tanning agents or hormones.

Particularly preferred embodiments of the present formulations are skin care lotions or creams used as an anti-aging product. To that end, the present formulations are combined with agents that are moisturizers, emollients or humectants. Examples of useful combinations are oils, fats, waxes, esters, fatty acid alcohols, fatty acid ethoxylates, glycols, sugars, hyaluronic acid and hyaluronates, dimethicone, cyclomethicone, and the like. Further examples can be found in the International Cosmetic Ingredient Dictionary, CTFA, Eighth Edition, 2000.

### Methods of Reducing the Signs of Aging

The methods taught herein, comprise administering or topically applying a skin beneficial amount of the composition of the present invention. The amount of the composition applied and the frequency of topical application to the skin may vary widely, depending upon the individual's needs and the level of regulation desired. A preferred method of cosmetically or pharmaceutically treating signs of aging in the skin, is via chronic topical application of a skin beneficial amount of the novel composition. It is well within the purview of the skilled artisan, such as a dermatologist or other health care provider, to regulate pharmaceutical dosages according to patient needs. The method of the present invention is suitable for daily use.

It is suggested as an example that topical application range from about once per week to about 2 or 3 times daily, preferably from about 5 times a week to about 3 times daily, most preferably about once or twice per day. The following examples further illustrate the invention, but the invention is not limited thereto.

### Example 1

### An Extraction Scheme for Menyanthes trifoliata Leaf Extracts

The following extraction scheme was useful in researching the properties of Menyanthes trifoliata leaf extracts. In the first step, an alcoholic solvent was applied to the dried leaves. Thereafter, the polarity of the solvents increases from the least polar, hexane to dichloromethane to ethyl acetate to the most polar, butanol. Ultimately, the components responsible for MMP-1, 2 and 9 inhibition reside in the alcoholic extract. However, additional extractions, as described below, were performed to further isolate the effective components. Some of those extracts (specifically, ethyl acetate and dichloromethane) were found to have suitable levels of the effective components. Thus, a number of solvents may be used to obtain Menyanthes trifoliata leaf extracts that inhibit MMPs-1, 2 and 9. Any of these extracts (alcoholic, ethyl acetate or dichloromethane) are suitable for compositions and methods of the present invention.

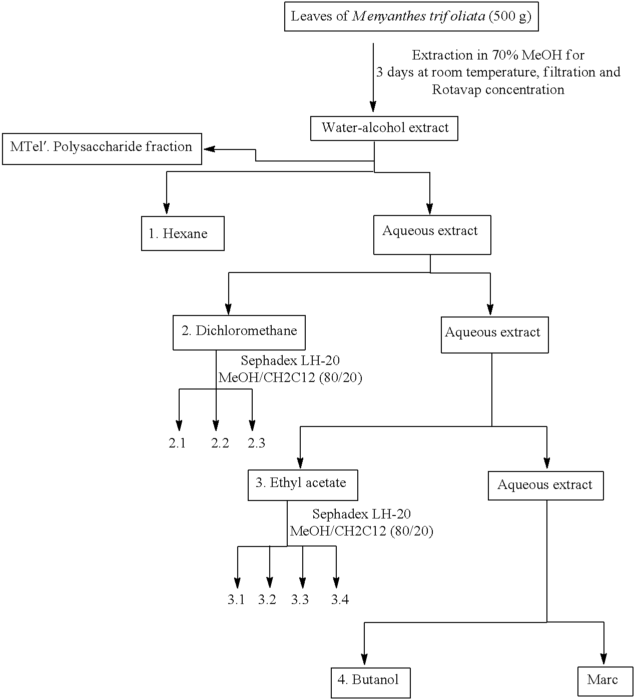

### Example 2

### In Vitro Inhibition of MMPs by Menyanthes trifoliata Leaf Extracts

Several extracts and sub-fractions from the leaves of Menyanthes trifoliata were prepared by liquid-liquid partitioning and fractionation on a Sephadex LH20 gel filtration column (see example 1) and evaluated for specific anti-MMP activity. In vitro specific inhibition of MMP-2 and MMP-9 activity was estimated with assay kits from Biomol®. Recombinant human MMP-1 enzyme may be obtained from any commercially available source. In table 1, MMP inhibition is expressed as IC₅₀ values, that is, the concentration of extract that results in a 50% reduction of the measured signal. Therefore, a lower value indicates a stronger MMP inhibition.

TABLE 1

| | inhibition expressed as IC₅₀ (μg/ml) against | | |
|---|---|---|---|
| Menyanthes trifoliata extract | MMP-1 | MMP-2 | MMP-9 |
| MTe1′. Polysaccharides | 240 | 240 | 240 |
| 1. Hexane | 115 | 142 | 88 |
| 2. Dichloromethane | 50 | § | § |
| 3. Ethyl acetate | 50 | 24 | 83 |
| 4. Butanol | § | 101 | 94 |

§: no activity measured

As seen from Table 1 above, the highest level of activity is found in the ethyl acetate and dichloromethane extracts. These two extracts are significantly more effective at MMP-1, 2, 9 inhibition. Because of its effectiveness at inhibiting all three MMPs, the ethyl acetate extract may be preferred, but dichloromethane extract may be used effectively and is within the scope of this invention. Of course, the alcoholic extract may also be used.

Sub-fractionation of the two crude extracts (ethyl acetate and dichloromethane) by separation on a Sephadex column results in extracts with even higher anti-metalloproteinase activity. The results are shown in Table 2.

TABLE 2

| | | inhibition expressed as IC₅₀ (μg/ml) | | |
|---|---|---|---|---|
| Menyanthes trifoliata | Sub- | against | | |
| extract | fraction | MMP-1 | MMP-2 | MMP-9 |
| 2. Dichloromethane | 2.1 | § | § | § |
| | 2.2 | 98 | 74 | 143 |
| | 2.3 | 37 | 40 | 40 |
| 3. Ethyl acetate | 3.1 | § | § | § |
| | 3.2 | 31 | 35 | 55 |
| | 3.3 | 15 | 15 | 32 |
| | 3.4 | 14 | 14 | 31 |

§: no activity measured

### Example 3

In order to determine the components responsible for the inhibition activity of the ethyl acetate and dichloromethane extracts, an HPLC compositional analysis of the extracts was performed. Table 3 shows amount of a component as a percent of the subfraction analyzed, on a weight basis. As can be seen in Table 3, phenolic acids, flavonoids and coumarins are the primary active components in ethyl acetate and dichloromethane extracts of Menyanthes trifoliata. Comparing tables 2 and 3, it is concluded that fractions with no or relatively low concentrations of phenolic acids, flavonoids and coumarins (fractions 2.1, 2.2 and 3.1), exhibit no or relatively poor MMP-1, 2, 9 inhibition activity. Conversely, those fractions with at least two of phenolic acids, flavonoids and coumarins exhibit significant inhibition activity.

TABLE 3

| | | FRACTIONS | | | | | | | | |
|---|---|---|---|---|---|---|---|---|---|---|
| Class | Component | 2 | 2.1 | 2.2 | 2.3 | 3 | 3.1 | 3.2 | 3.3 | 3.4 |
| Phenolic acids | protocate-chuic acid | * | * | * | <0.1 | 0.4 | * | 0.4 | 0.3 | <0.1 |
| | p-hydroxy | * | * | * | * | * | * | 0.9 | * | * |
| | benzoic acid | | | | | | | | | |
| | ferulic acid | * | * | * | 1.6 | * | * | * | * | * |
| Flavonoids | quercetin | 0.3 | * | * | * | 2.9 | * | * | * | 96 |
| | iso-quercitrin | * | * | * | * | 4.2 | <0.1 | * | 19 | 0.1 |
| | Rutin | 0.2 | * | * | 4.8 | 1.8 | * | 13.1 | 1.3 | 0.4 |
| Coumarins | Scopoletin | NT | NT | NT | 0.22 | * | * | * | * | * |
| | Scoparone | NT | NT | NT | 0.03 | * | * | * | * | * |

* not detectable
NT—not tested

### Example 4

### In Vitro Inhibition of MMPs by Selected Standards

To further understand which agents may be contributing to the MMP-1, 2, 9 inhibition activity, standards of the different phenol acids, flavonoids and coumarins, identified in Menyanthes trifoliata extracts, were tested for their in vitro inhibition of MMPs-1, 2 and 9. In vitro specific inhibition of MMP-2 and MMP-9 activity is estimated with assay kits from Biomol®. Recombinant human MMP-1 enzyme is obtained from any commercially available source. Results are summarized in Table 4.

TABLE 4

| | | inhibition expressed as | | |
|---|---|---|---|---|
| | | IC₅₀ (μg/ml) against | | |
| Class | Component | MMP-1 | MMP-2 | MMP-9 |
| Phenolic acids | p-hydroxy-benzoic acid | § | § | § |
| | ferulic acid | 28 | 22 | 20 |
| Flavonoids | Quercetin | 21 | 10 | 19 |
| | Iso-quercitrin | 23 | 10 | 92 |
| | Rutin | 40 | 28 | 43 |
| Coumarins | Scopoletin | 15 | 18 | 25 |
| | Scoparone | 15 | 18 | 10 |

§: no activity measured

As seen in Table 4, ferulic acid, but not p-hydroxy-benzoic acid, shows strong in vitro MMP-1, 2 and 9 inhibition. Flavonoids (quercetin, iso-quercetrin and rutin) and coumarins (scopoletin and scoparone) all show strong anti-metalloproteinase activity.

### Example 5

### Peroxynitrite Scavenging Activity of Menyanthes trifoliata Leaf Extracts

Several extracts and sub-fractions from the leaves of Menyanthes trifoliata were prepared by liquid-liquid partitioning and fractionation on a Sephadex column (see Example 1) and evaluated for peroxynitrite scavenging. Assay was performed with the ABEL® peroxynitrite antioxidant test kit with Pholasin®. Results are summarized in Table 5.

TABLE 5

| Menyanthes trifoliata | | Peroxynitrite scavenging |
|---|---|---|
| extract | Sub-fraction | (IC₅₀ as μg/ml) |
| 2. Dichloromethane | 2.3 | <1.2 |
| 3. Ethyl acetate | 3 | 2.1 |
| | 3.2 | <1 |
| | 3.3 | <1 |
| | 3.4 | 2.1 |

### Example 6

### Peroxynitrite Scavenging Activity of Selected Set of Standards

Standards of different phenolic acids and flavonoids, identified in Menyanthes trifoliata extracts, were tested for their in vitro peroxynitrite scavenging activity. Assay was performed with the ABEL® peroxynitrite antioxidant test kit with Pholasin®. Results are summarized in Table 6.

TABLE 6

| | | Peroxynitrite scavenging |
|---|---|---|
| Class | Component | (IC₅₀ as μg/ml) |
| Phenolic acids | p-hydroxy-benzoic acid | § |
| | ferulic acid | 3.1 |
| | Protocatechuic acid | 0.6 |
| Flavonoids | Quercetin | 1.6 |
| | Iso-quercitrin | 1 |
| | Rutin | 1.8 |

§: no activity measured

Ferulic acid and protocatechuic acid but not p-hydroxy-benzoic acid are strong scavengers of peroxynitrite. Flavonoids (quercetin, iso-quercetrin and rutin) all show strong peroxynitrite scavenging activity.

The data show that the ethyl acetate extracts are more potent inhibitors of MMPs than the dichloromethane extracts, although the dichloromethane extracts are quite useful for the purpose. On the other hand, the two extracts are similar in their ability to scavenge peroxynitrite. Either extract or a combination may be used effectively to practice the present invention. Of course, the alcoholic extract may also be used.

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

What is claimed is:

1. A cosmetic composition comprising:
a skin-beneficial amount of actives identified in Menyanthes trifoliata leaf, wherein the actives are inhibitors of one or more of MMP-1, 2 or 9 and/or scavengers of peroxynitrite; and
a cosmetically acceptable vehicle, with the condition that composition contains no Polygonum aviculare and/or no extract of horse chestnut.

2. The composition of claim 1 wherein the actives are flavonoids selected from the group consisting of quercetin, iso-quercetrin, rutin and mixtures thereof.

3. The composition of claim 1 wherein the actives are phenolic acids selected from ferulic acid, protochatechuic acid and mixtures thereof.

4. The composition of claim 1 wherein the actives are coumarins selected from the group consisting of scoparone, scopoletin and mixtures thereof.

5. The composition of claim 1 which comprises 0.001% to 15% by weight of the actives.

6. The composition of claim 1 further comprising a sunscreen selected from the group consisting of water soluble sunscreens, oil soluble sunscreens, inorganic sunscreens, and organic sunscreens.

7. A cosmetic composition comprising:
a skin-beneficial amount of Menyanthes trifoliata leaf extract, wherein the extract inhibits one or more of MMP-1, 2 or 9 and/or scavenges peroxynitrite; and
a cosmetically acceptable vehicle, with the condition that composition contains no Polygonum aviculare and/or no extract of horse chestnut.

8. The composition of claim 7 which comprises 0.001% to 20% by weight of one or more Menyanthes trifoliata leaf extracts.

9. The composition of claim 8 wherein at least some of the one or more extracts are an alcoholic extract, an ethyl acetate extract, a dichloromethane extract or mixtures thereof.

10. The composition of claim 9 wherein the extract of Menyanthes trifoliata comprises skin-beneficial amounts of phenolic acids, flavonoids, and coumarins.

11. The composition of claim 10 wherein the flavonoids are selected from the group consisting of iso-quercetrin and rutin and mixtures thereof.

12. The composition of claim 10 wherein the phenolic acids are selected from ferulic acid and protochatechuic acid.

13. The composition of claim 10 wherein the coumarins are selected from scoparone and scopoletin.

14. The composition of claim 7 further comprising a sunscreen selected from the group consisting of water soluble sunscreens, oil soluble sunscreens, inorganic sunscreens and organic sunscreens.

15. A method of reducing the signs of photoaging on the skin comprising a step of applying a composition comprising:
a skin-beneficial amount of actives identified in Menyanthes trifoliata leaf, wherein the actives are inhibitors of one or more of MMP-1, 2 or 9 and/or scavengers of peroxynitrite; and
a cosmetically acceptable vehicle, with the condition that composition contains no Polygonum aviculare and/or no extract of horse chestnut.

16. The method of claim 15 wherein the actives are flavonoids selected from the group consisting of iso-quercetrin and rutin and mixtures thereof.

17. The method of claim 15 wherein the actives are phenolic acids selected from ferulic acid and protochatechuic acid.

18. The method of claim 15 wherein the actives are coumarins selected from scoparone and scopoletin.

19. The method of claim 15 wherein the composition comprises 0.001% to 15% by weight of the actives

20. The method of claim 15 wherein the composition comprises from 0.001% to 20% by weight of a Menyanthes trifoliata leaf extract.
